(19) United States
(12) Patent Application Publication
(10) Pub. No.: US 2004/0030336 A1
(43) Pub. Date: Feb. 12, 2004

(54) Anterior cervical spine stabilization method and system

(76) Inventor: Rohit Kumar Khanna, Daytona Beach, FL (US)

Correspondence Address: ROHIT K. KHANNA, SUITE 460, 311 NORTH CLYDE MORRIS BLVD, DAYTONA BEACH, FL, 32114, US

(21) Appl. No.: 10/213,159

(22) Filed: Aug. 6, 2002

(51) Int. Cl. A61B 17/58

(52) U.S. Cl. 606/61

(57) ABSTRACT

Spinal column stabilization method and devices are described. The devices comprise of a plate, bone screws, a distractor/compressor, and locking screws. Variations in the locking screws and plates provide for fixed or variable angle screw placement. Several variations of the bone and locking screws with capabilities of expansion of the bone screw head as well as the tips are disclosed. The bone screws, prior to placement of the locking screws, have a smaller diameter than the bone screw holes of the plate, which allows their placement prior to the plate, and therefore, the capability of also being used as compression and/or distraction screws. The locking screws expand the bone screw head securing it to the plate and also prevent screw back out.

### BACKGROUND OF THE INVENTION

The present invention is an apparatus for use in fixation of the cervical spine. The invention provides for cervical distraction, bone graft compression, as well as retaining cervical vertebrae in a desired spatial relationship.

There are many cervical spine plating systems that have been recently developed. The utility of such plates is to provide immediate spine stability until the interbody bone fusion has occurred. Variabilities in the currently available anterior cervical plates include their thickness, fixed or variable angle bone screws, and bi-cortical or unicortical screws with various locking mechanisms.

With the current surgical technique, prior to placement of the cervical plate, most surgeons typically place removable distraction screws in the vertebral bodies adjacent to the levels being fused to facilitate exposure, correct kyphosis, allow distraction and/or placement of a bone graft. Subsequently after the interbody bone graft placement, the distraction screws are removed and the cervical plate and screws are placed in the same vertebral bodies. The potential pitfall with this technique is the limited size of the cervical vertebral body and the disruption in its integrity sustained by placing a distraction screw that is subsequently removed, thus allowing for a weaker purchase of the cervical plate anchoring screws placed adjacent to this vacant screw hole. These distraction screws also do not allow compression of the bone graft if needed during the cervical plate placement.

The present cervical plating system allows for the use of vertebral body screws that allow distraction as well as plate anchoring and bone graft compression without having to remove the screws.

### SUMMARY OF THE INVENTION

The present invention relates an anterior cervical fixation system, which includes a plate with multiple screw holes, bone screws, and locking screws. The bone screws provide for a plate to be placed after the screw has been implanted. This allows the same bone screws to be used for cervical vertebral body distraction for surgery prior to the plate placement. These screws also allow for placement of the plate and compression of the bone graft if so desired. A locking screw with a superior shoulder is engaged into the bone screw to secure it to the cervical plate. In another embodiment, the plate has elongated cephalad screw holes, which provide for variable screw angle placement as well as bone settling during the fusion.

A novel bone screw is disclosed with an expandable head following engagement by a locking screw with an expanded head so as to secure the bone screw to the plate. In another embodiment, the bone screw has an expandable head and tip which expands after engagement with a locking screw with an expanded head and elongated tip. The expanded bone screw head following placement of the locking screw secures the bone screw to the plate whereas the expansion of the bone screw tip provides for a more secure bone purchase preventing pulling out of the bone screw.

In another embodiment, the superior bone screw holes in the plate have a unique socket type configuration at the edges so as to allow the ball shaped configurations in the shoulder of the locking screw to settle in the sockets. This allows the bone screw to swivel relative to the plate thereby providing dynamic movement between the plate enabling bone settling during the fusion.

In another embodiment, the locking screw shoulder has a triangular configuration which constricts the corresponding circumferential ridge around the screw holes of the plate into the screw once the locking screw is engaged into the bone screw, thereby providing another venue to secure the bone screw to the plate.

The uniqueness of the current invention is in the low profile plate and screw design that allows a bone screw to be placed prior to the plate placement. This same bone screw can be used as a distraction or compression screw as well as cervical plate anchoring screw. This eliminates the need for placement of a distracting screw that is subsequently removed, thereby maintaining the vertebral body integrity, strength, and space needed for placement of the cervical plate bone screws in an already very limited space.

In yet another embodiment, the cervical plate has a low profile with elongated central screw holes at each end that allow for smaller diameter screws to be placed which are used for cervical distraction to improve exposure, correct the cervical alignment, as well as placement of the bone graft for fusion. These same screws can be used for compression of the bone graft after the cervical plate is slid over them. The advantage of this system is that the distraction/compression screws provide for a plate to be placed after the screw has been implanted. This screw also allows for placement of the plate and compression of the bone graft if so desired. The variable length central distraction screw holes allow for compression prior to placement of the bone screws. The smaller diameter distraction screws are removed after the bone screws are placed.

### BRIEF DESCRIPTION OF THE DRAWINGS

A better understanding of the present invention will be had after a reading of the following detailed description taken in conjunction with the accompanying drawings wherein:

FIG. 1 is a top view of one embodiment of the cervical plate

FIG. 2 is a cross-sectional side view of the plate taken along line A in FIG. 1

FIG. 3 is a cross-sectional view of the plate taken along line B in FIG. 1

FIG. 4 is a top view of another embodiment of the cervical plate

FIG. 5 is a cross-sectional side view of the plate taken along line C in FIG. 4

FIG. 6 is a cross-sectional side view of the plate taken along line C in FIG. 4 with bone screw in the superior hole

FIG. 7 is a cross-sectional side view of another embodiment of the cervical plate

FIG. 8 is a cross-sectional view of the plate in FIG. 7 with a bone screw in place

FIG. 9 is a cross-sectional side view of the plate in FIG. 7 with bone screws in place

FIG. 10 is a cross-sectional side view of the plate in FIG. 7 with bone screws in place

FIG. 11 is a top view of another embodiment of the cervical plate

FIG. 12 is a top view of another embodiment of the cervical plate

FIG. 13 is a side view of one embodiment of the locking screw

FIG. 14 is a cross-sectional side view of one embodiment of the bone screw

FIG. 15 is a cross-sectional side view of the bone screw with the locking screw engaged

FIG. 16 is a side view of another embodiment of the locking screw

FIG. 17 is a side view of the bone screw with the locking screw engaged

FIG. 18 is a side view of another embodiment of the locking screw

FIG. 19 is a cross-sectional side view of another embodiment of the bone screw

FIG. 20 is a cross-sectional side view of the bone screw with the locking screw engaged

FIG. 21 is a side view of another embodiment of the locking screw

FIG. 22 is a side view of the of the bone screw with the locking screw engaged

FIG. 23 is a side view of another embodiment of the locking screw

FIG. 24 is a side view of another embodiment of the bone screw

FIG. 25 is a cross-sectional side view of the bone screw taken along line D in FIG. 24

FIG. 26 is a cross-sectional side view of the bone screw with the locking screw engaged

FIG. 27 is a side view of the bone screw with the locking screw engaged

FIG. 28 is a side view of another embodiment of the locking screw

FIG. 29 is a side view of the bone screw with the locking screw in place

FIG. 30 is a cross-sectional side view of the spine with the bone screws in place

FIG. 31 is side view of the distractor

FIG. 32 is a cross-sectional side view of the spine with the arms of the distractor in place on the bone screws

FIG. 33 is a close-up cross-sectional side view of the distractor tip engaged with the bone screw

FIG. 34 is a cross-sectional side view of the spine

FIG. 35 is a cross-sectional side view of the spine

FIG. 36 is a top view of another embodiment of the cervical plate

FIG. 37 is a top view of another embodiment of the cervical plate

FIG. 38 is a top view of another embodiment of the cervical plate

FIG. 39 is a top view of another embodiment of the cervical plate

FIG. 40 is a cross-sectional side view of the cervical plate with a screw in place taken along line E in FIG. 37

FIG. 41 is a side view of another embodiment of the locking screw

FIG. 42 is a cross-sectional side view of a bone screw

FIG. 43 is a side view of the bone screw with the locking screw engaged

FIG. 44 is a cross-sectional side view of the bone screw with locking screw engaged

FIG. 45 is a side view of another embodiment of the locking screw

FIG. 46 is a cross-sectional side view of a bone screw

FIG. 47 is a cross-sectional side view of the bone screw with the locking screw engaged

FIG. 48 is a side view of another embodiment of the locking screw

FIG. 49 is a cross-sectional side view of a bone screw

FIG. 50 is a cross-sectional side view of the bone screw with the locking screw engaged

### DETAILED DESCRIPTION OF THE DRAWINGS

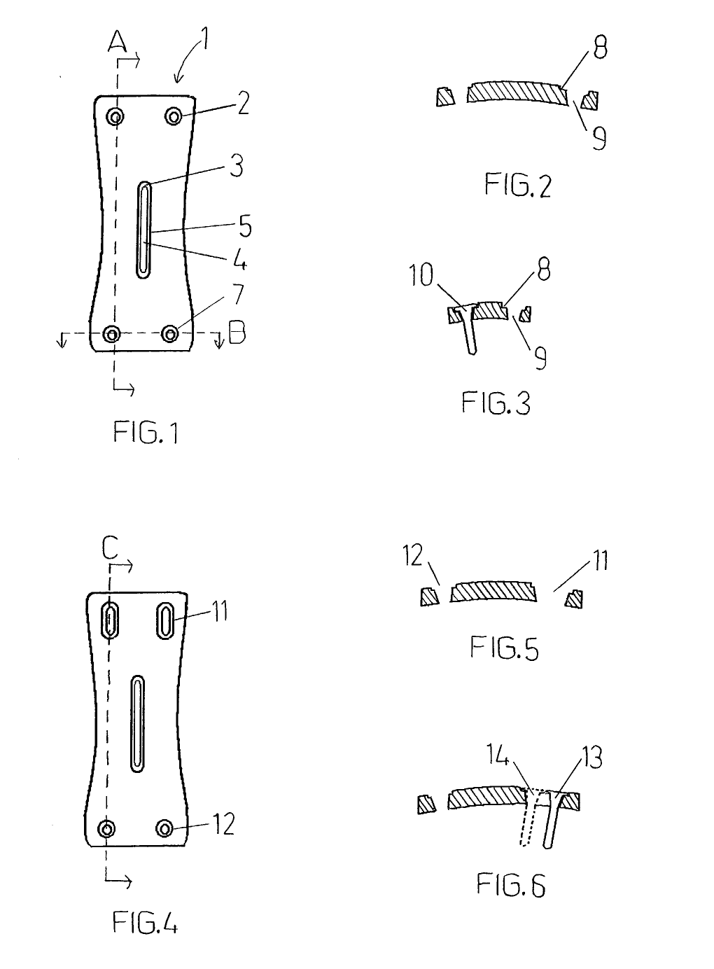

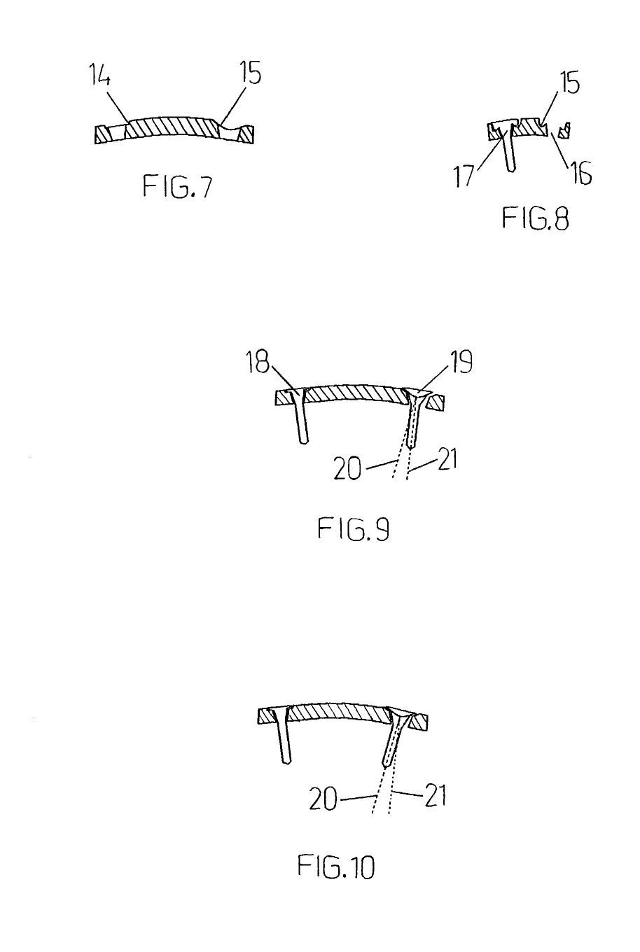

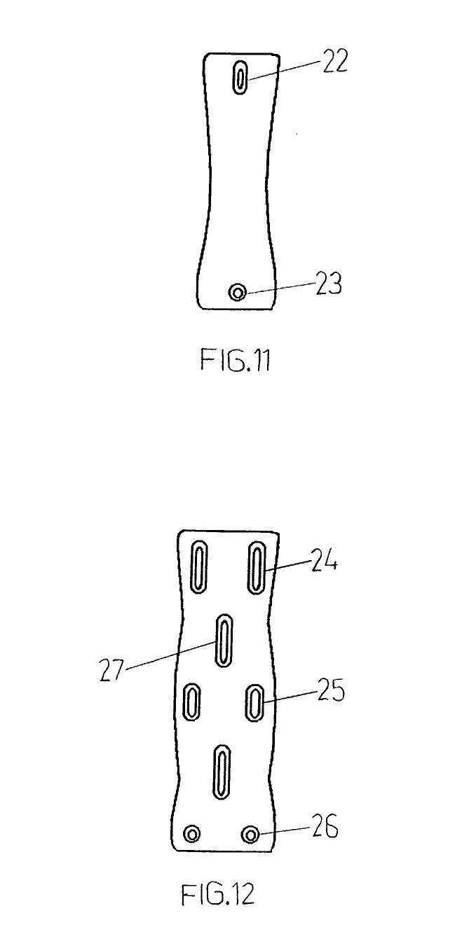

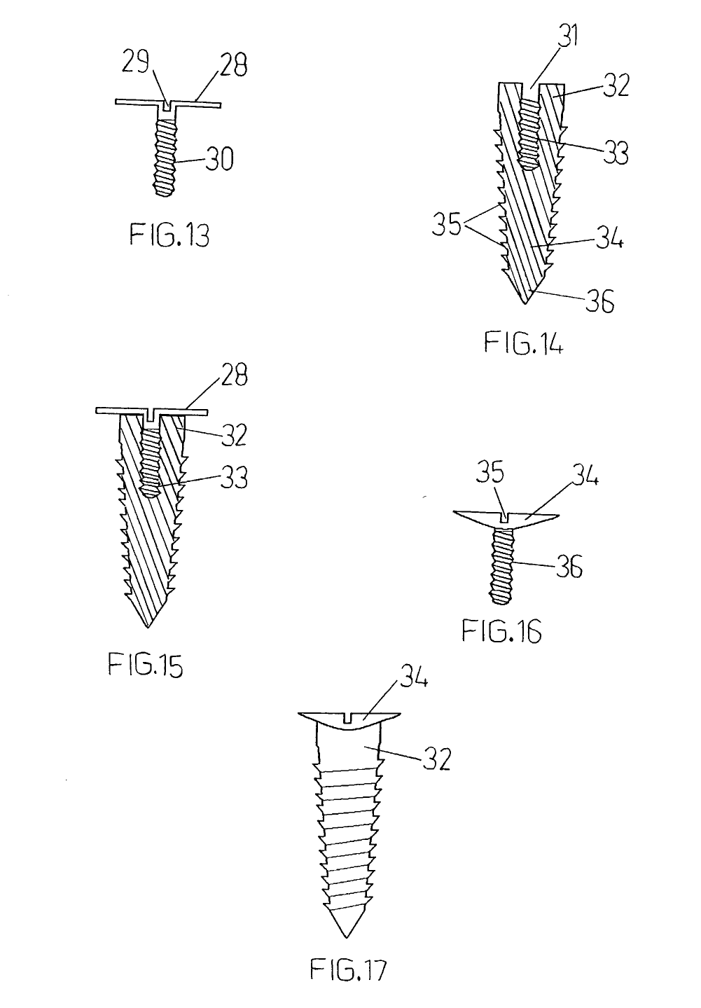

In one embodiment as shown in FIGS. 1-3, the cervical plate 1 has superior bone screw holes 2 and inferior bone screw holes 7 with a central elongated bone screw hole 5. The bone screw holes have a circumferential depression 8 surrounding the bone screw hole 9 which provide for a fixed angle bone screw 10 placement. The central elongated bone screw hole 5 also has a circumferential depression 3 surrounding the hole 4.

In another embodiment as shown in FIGS. 4-6, the cervical plate has superior elongated bone screw holes 11 and inferior bone screw holes 12. The elongated superior bone screw holes 11 allow for dynamic screw movement and bone settling during fusion whereas the inferior bone screw holes 12 allow for a fixed bone screw placement. FIG. 6 illustrates the bone screw translation during the fusion process from position 13 to position 14.

In another embodiment as shown in FIGS. 7 and 8, the cervical plate has a variation in the superior bone screw holes with a convexity 15 at either sides of the bone screw hole 16 which allows the bone screw to swivel thereby allowing for variable angle bone screw placement 17 and bone settling during fusion. The inferior bone screw holes have a circumferential depression 14 surrounding the bone screw hole, which provides for a fixed angle bone screw placement. FIGS. 9 and 10 illustrate the variable angle bone screw 19 and the fixed angle bone screw 18 in place. The settling of the screw from position 21 to 20 during the fusion process is possible by the variable angle placement allowed by the superior screw hole and screw design.

In another embodiment as shown in FIG. 11, the cervical plate has one superior bone screw hole 22 and one inferior bone screw hole 23. In a variation of this plate, the superior bone screw hole 22 can be elongated to allow for dynamic movement and settling during bone fusion

In another embodiment as shown in FIG. 12, the cervical plate is designed for a multiple level vertebral body stabilization. The superior bone screw holes 24 and middle bone screw holes 25 are elongated to allow for bone settling whereas the inferior bone screw holes 26 are for a fixed angle bone screw placement. The plate also has central bone screw holes 27.

Several embodiments of the bone screws as well as the corresponding locking screws are described henceforth.

In one embodiment as shown in FIG. 13, the locking screw has a shoulder 28, a threaded shaft 30, and a recess 29 for screw tightening. The bone screw, as shown in FIG. 14, has a threaded locking hole 33 for placement of the locking screw. The head of the bone screw 32 expands slightly when the locking screw is in place in the center hole 31 of the bone screw. The bone screw also has threads 35 and screw tip 36 that can also be adjusted for a self-drilling screw variation. FIG. 15 illustrates the bone screw engaged with the locking screw. The superior shoulders 28 of the locking screw as well as expansion of the bone screw head 32 after placement of the locking screw with engagement to the threaded locking hole 33 of the bone screw, secures the screw on to the cervical plate.

The locking screw as shown in FIG. 16, is specifically designed to allow for placement at a variable angle as well as settling during the fusion process. It has a superior shoulder 34 with four convex edges that settle on the corresponding convexity 15 on the superior bone screw holes of the cervical plate as shown in FIGS. 8-10. The locking screw also has a threaded shaft 36 and recess 35 that allows placement of the screw to the corresponding threaded hole 33 of the bone screw. The bone screw is the same as illustrated in FIG. 14. The bone screw with locking screw in place is shown in FIG. 17. The convex shoulder 34 allows variable angle placement that along with bone screw head expansion 32 secures the screw to the plate.

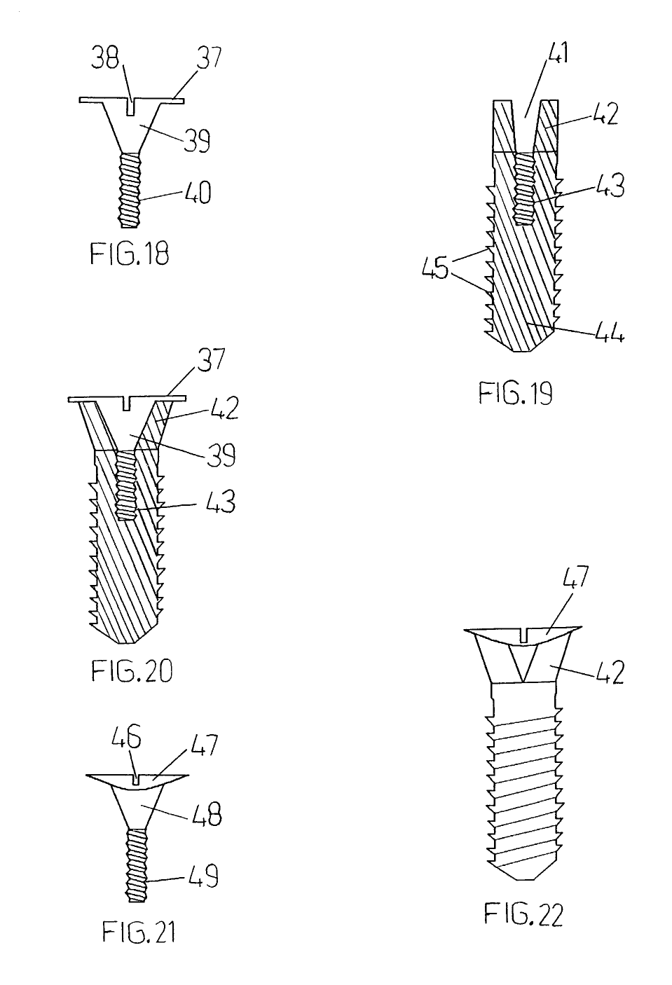

In another embodiment of the screw as shown in FIGS. 18-20, the bone screw as illustrated in FIG. 19 has an expandable head 42 as well as a central opening 41 and a threaded center hole 43 that allows for placement of the locking screw. The bone screw also has threads 45 and screw tip 44 that can also be adjusted for a self-drilling screw variation. The locking screw as illustrated in FIG. 18, has a superior shoulder 37, an expanded head 39 with a recess 38 and a threaded shaft 40. Following placement of the locking screw into the bone screw as illustrated in FIG. 20, the expanded head 39 of the locking screw as it secures to the threaded hole 43 of the bone screw, expands the head 42 of the bone screw thereby securing the bone screw to the cervical plate. The shoulders 37 of the locking screw further secure the screw to the plate. FIGS. 21 and 22 illustrate the variable angle screw with an expandable head design. The bone screw is the same as illustrated in FIG. 19 but the locking screw as shown in FIG. 21 has a superior shoulder 47 with four convex edges that settle on the corresponding convexity 15 on the superior bone screw holes of the cervical plate as shown in FIGS. 8-10. The locking screw also has an expanded head 48, a threaded shaft 49 and recess 46 that allows placement of the screw to the corresponding threaded hole 43 of the bone screw. FIG. 22 illustrates the bone screw with the locking screw in place. The convex shoulder 47 allows variable angle placement, which along with bone screw head expansion 42 secures the screw to the plate.

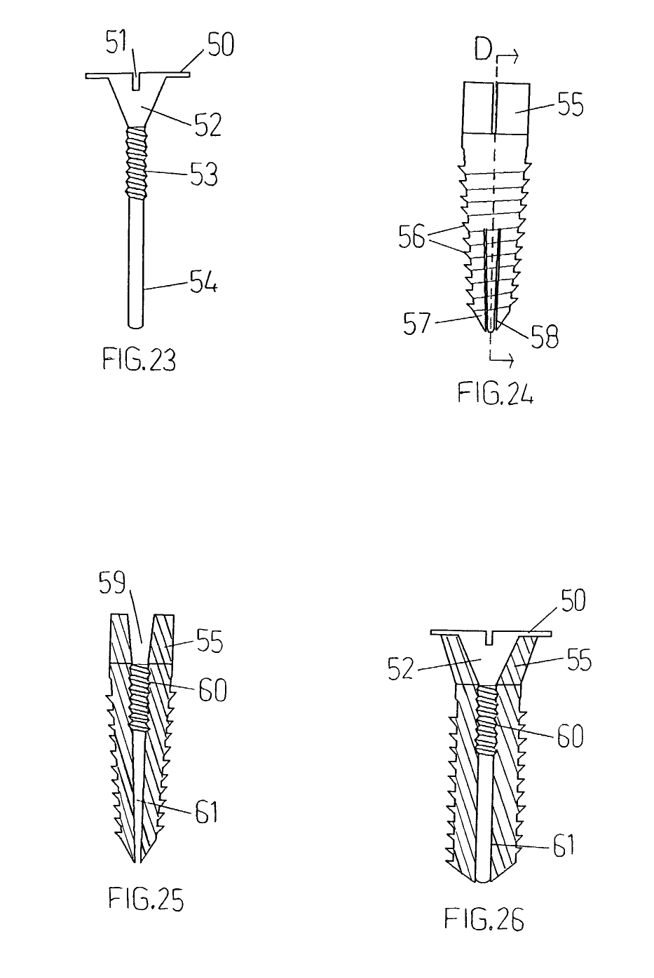

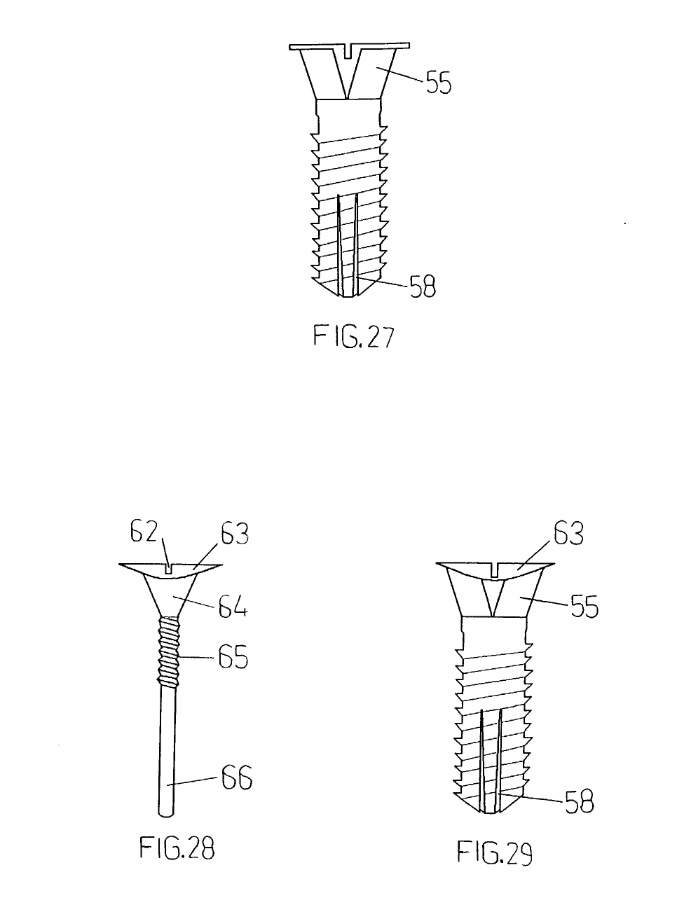

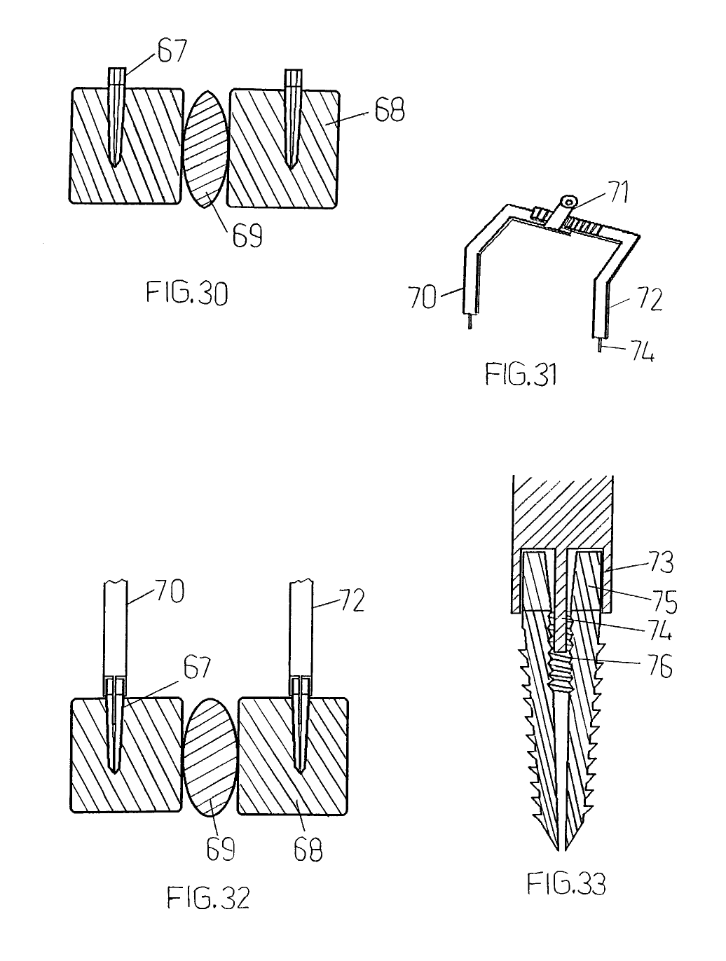

In another embodiment of the screw as shown in FIGS. 23-27, the bone screw shown in FIGS. 24 and 25, has an expandable head 55 as well as a central opening 59, a threaded center hole 60, and an elongated tip hole 61. The bone screw also has threads 35 and screw tips 57 and 58 with circumferential openings that expand when the locking screw is in place. The locking screw as shown in FIG. 23 has a superior shoulder 50, an expanded head 52 with a recess 51, a threaded shaft 53, and an expanded tip 54. Following placement of the locking screw into the bone screw the head 55 and the tip hole 61 expand with widening of the distal bone screw openings 58 as shown in FIGS. 26 and 27, thereby securing the bone screw to the cervical plate and the vertebral body. FIGS. 28 and 29 illustrate the variable angle screw with an expandable head design. The bone screw is the same as illustrated in FIGS. 24 and 25 but the locking screw as shown in FIG. 28 has a superior shoulder 63 with four convex edges that settle on the corresponding convexity 15 on the superior bone screw holes of the cervical plate as shown in FIGS. 7-10. The locking screw also has an expanded head 64, an elongated tip 66, a threaded shaft 65 and recess 62 that allows placement of the screw to the corresponding threaded hole 60 of the bone screw. FIG. 29 illustrates the bone screw with the locking screw in place. The convex shoulder 63 allows the screw to swivel during the fusion process with variable angle screw placement as well as preventing the cervical plate from backing out. The expanded screw head 55 also secures the screw to the cervical plate and the expanded screw tip 58 secures the screw to the vertebral body.

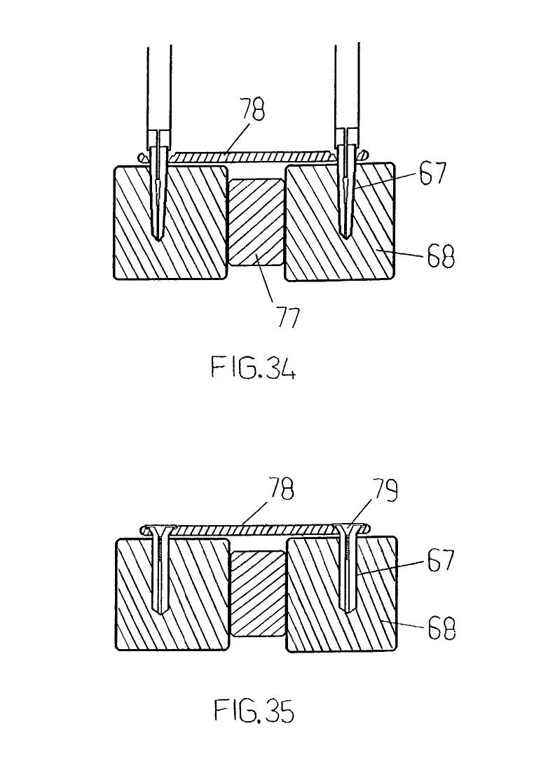

The method of cervical stabilization is illustrated in FIGS. 30 to 35. FIG. 30 shows the bone screws 67 placed in the vertebral bodies 68 between the disc 69. The distractor/compressor is illustrated in FIG. 31. The distractor has cannulated tubes 70 and 72 with a pin 74 in the center and a locking mechanism 71. As shown in FIGS. 32 and 33, after the distractor is placed on the bone screws 67, the disc space 69 is increased to improve exposure. The central pin 74 of the distraction tube 70 and 72 fits into the central hole 76 of the bone screw 67 and the cannula 73 of the distraction tube surrounds the bone screw head 75 so as to secure the distractor to the bone screws and use them for distraction or compression. FIG. 34 illustrates the placement of the bone graft 77 following the discectomy. The cervical plate 78 is then placed sliding through the bone screws 67. In FIG. 35, the distractor/compressor has been removed, the bone screws 67 advanced further into the vertebral bodies 68 and the locking screws 79 have been placed to secure the bone screws 67 to the cervical plate 78.

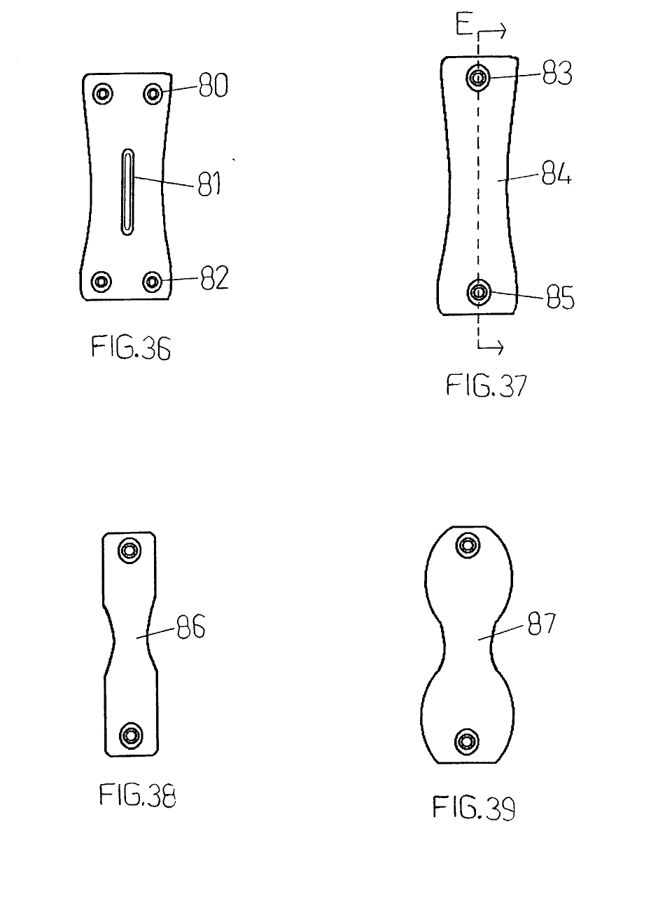

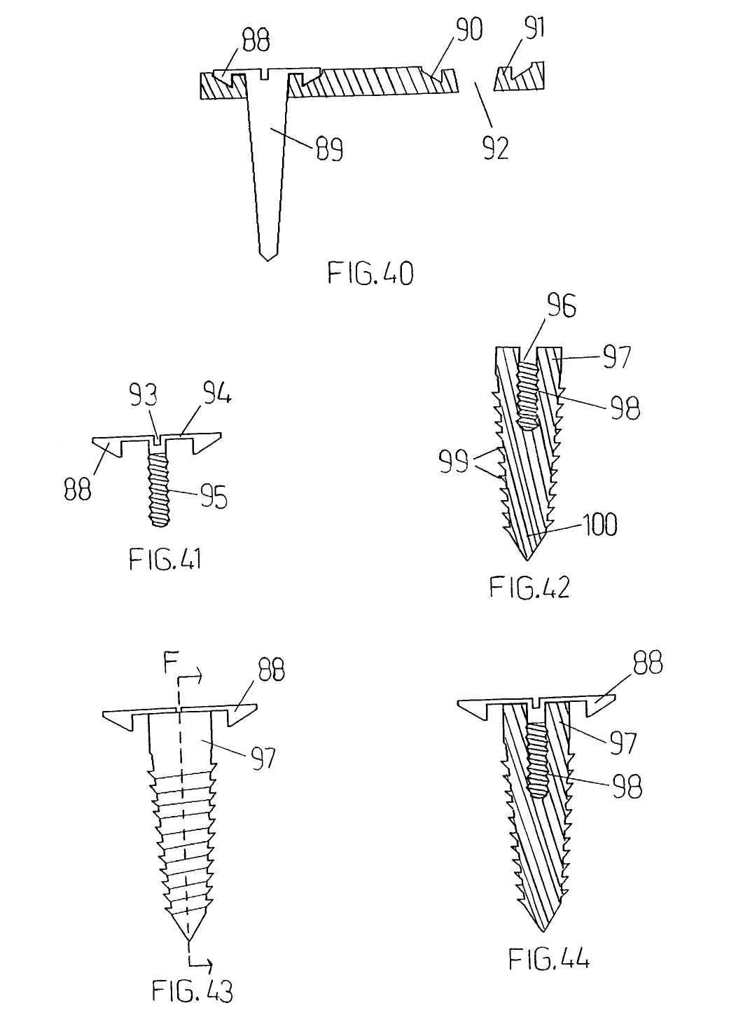

Several other embodiments of the cervical plate with another variation in the bone screw hole design are shown in FIGS. 36 to 39. As illustrated in FIG. 36, the cervical plate has superior bone screw holes 80, inferior bone screw holes 82, and a central elongated bone screw hole 81. FIGS. 37, 38, and 39 illustrate cervical plates with a single superior 83 and inferior 85 bone screw holes with several design variations and tapered center 84, 86, and 87 to allow better visualization of the disc space. As shown in FIG. 40, the bone screw hole 92 has a circumferential ridge 91 surrounded by a circumferential triangular shaped depression 90. Following placement of the bone screw 89 with the locking screw in place, the screw head circumferential triangular ridge 88 fits in the corresponding depression 90 in the plate that compresses the circumferential ridge 91 against the screw head.

In one embodiment of the screw design as shown in FIG. 41, the locking screw has a circumferential shoulder 94 with a triangular circumferential ridge 88, a threaded shaft 95, and a recess 93 for screw tightening. The bone screw as shown in FIG. 42 is the same as the one in FIG. 14. The bone screw has a threaded locking hole 98 for placement of the locking screw. The head of the bone screw 97 expands slightly when the locking screw is in place in the center hole 96 of the bone screw. The bone screw also has threads 99 and screw tip 100 that can also be adjusted for a self-drilling screw variation. FIGS. 43 and 44 illustrate the bone screw with the locking screw in place. The superior shoulders 88 of the locking screw as well as expansion of the bone screw head 97 after placement of the locking screw with engagement to the threaded locking hole 98 of the bone screw, secures the screw on to the cervical plate.

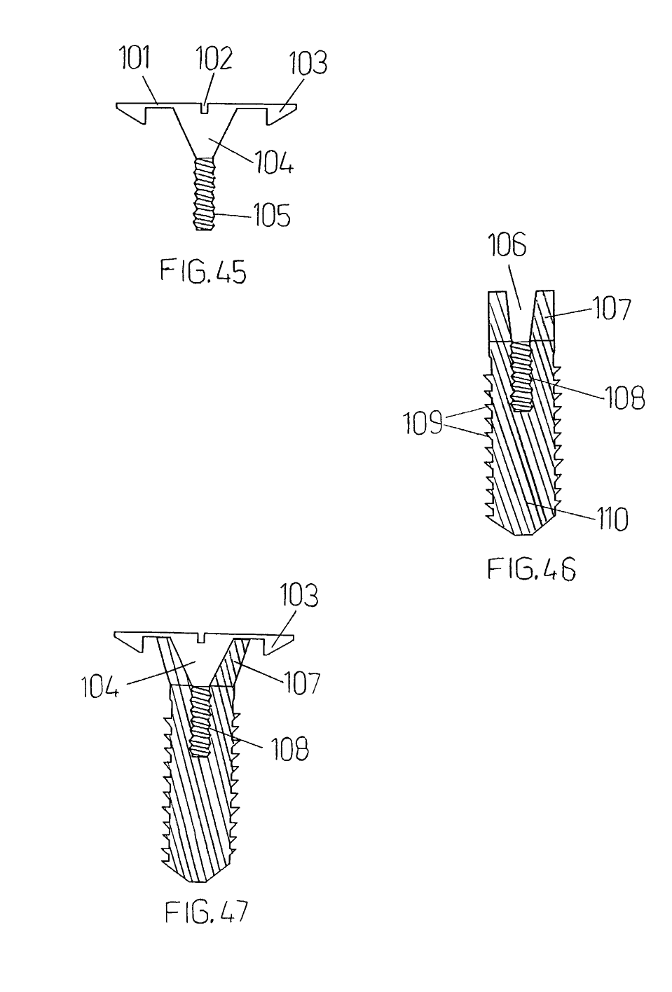

In another embodiment of the screw as shown in FIG. 45, the locking screw has a superior shoulder 101 with a triangular circumferential ridge 103, an expanded head 104 with a recess 102, and a threaded shaft 105. The bone screw as shown in FIG. 46 is the same as in FIG. 19. The bone screw has an expandable head 107 as well as a central opening 106 and a threaded center hole 108 that allows for placement of the locking screw. The bone screw also has threads 109 and screw tip 110 that can also be adjusted for a self-drilling screw variation. Following placement of the locking screw into the bone screw as illustrated in FIG. 47, the expanded head 104 of the locking screw as it secures to the threaded hole 108 of the bone screw, expands the head 107 of the bone screw thereby securing the bone screw to the cervical plate. The shoulders 103 of the locking screw further secure the screw to the plate.

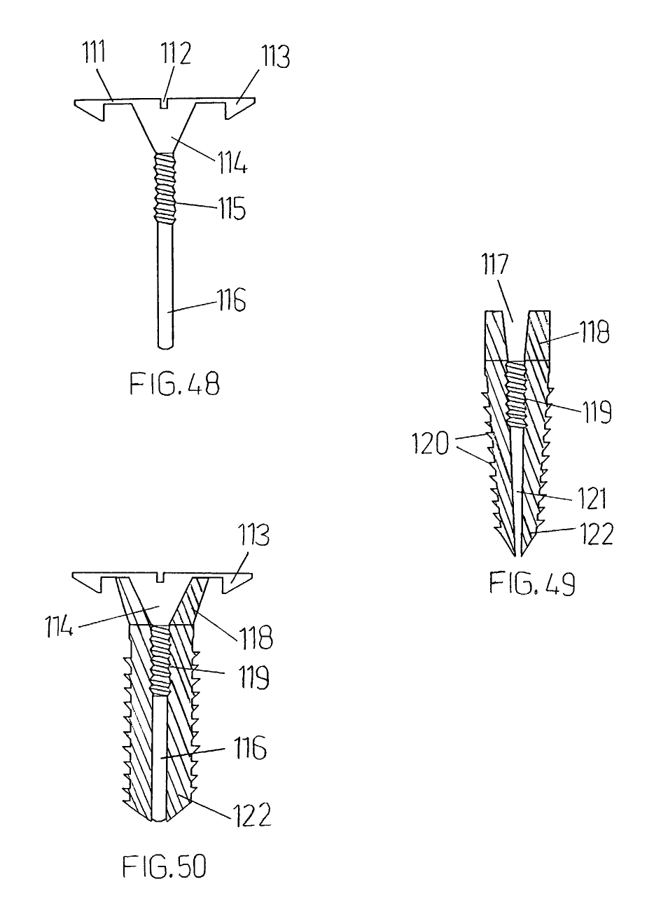

In another embodiment as shown in FIG. 48, the locking screw has a superior shoulder 111 with a triangular circumferential ridge 113, an expanded head 114 with a recess 112, a threaded shaft 115, and an elongated tip 116. The bone screw as shown in FIG. 49 is the same as in FIG. 25. The bone screw has an expandable head 118 as well as a central opening 117, a threaded center hole 119, and an elongated tip hole 121. The bone screw also has threads 120 and screw tip 122 that expands when the locking screw is in place. Following placement of the locking screw into the bone screw as shown in FIG. 50, the bone screw head 118 and the tip 122 is expanded by the expanded head 114 and elongated tip 116 of the locking screw as it secures to the threaded hole 119. The shoulder 113 along with the screw head and tip expansion secure the bone screw to the cervical plate and the vertebral body.

While the present invention has been described in conjunction with preferred embodiments and methods, it is intended that the description and accompanying drawings shall be interpreted as only illustrative of the invention. It is evident that those skilled in the art may make numerous uses and modifications of and departures from the specific embodiments described herein without departing from the inventive concept.

### REFERENCES

U.S. Patent Documents

| | | |
|---|---|---|
| 2699774 | January, 1955 | Livingston. |
| 3659595 | May, 1972 | Haboush. |
| 3741205 | June, 1973 | Markolf et at. |
| 3779240 | December, 1973 | Kondo. |
| 3842825 | October, 1974 | Wagner. |
| 4388921 | June, 1983 | Sutter et at. |
| RE31628 | July, 1984 | Allgower et at. |
| 4484570 | November, 1984 | Sutter et at. |
| 4488543 | December, 1984 | Tornier. |
| 4493317 | January, 1985 | Klaue. |
| 4503848 | March, 1985 | Caspar et al. |
| 4794918 | January, 1989 | Wolter. |
| 4913134 | April, 1990 | Luque. |
| 5041113 | August, 1991 | Biedermann et al. |
| 5084049 | January, 1992 | Asher et at. |
| 5129899 | July, 1992 | Small et at. |
| 5176679 | January, 1993 | Lin. |
| 5180381 | January, 1993 | Aust et at. |
| 5261910 | November, 1993 | Warden et at. |
| 5531746 | July, 1996 | Errico et al. |
| 5549612 | August, 1996 | Yapp et al. |
| 5607426 | March, 1997 | Ralph et at. |
| 5676666 | October, 1997 | Oxland et at. |
| 5713900 | February, 1998 | Benzel et at. |
| 5954722 | September, 1999 | Bono. |
| 6193721 | February, 2001 | Michelson. |
| 6228085 | May, 2001 | Theken et al. |
| 6258089 | July, 2001 | Campbell et al. |
| 6328738 | December, 2001 | Suddaby. |

What is claimed is:

1. A cervical spine stabilization apparatus comprising:
a fixation plate with a longitudinal axis and two or more bone fastener holes for engaging at least two vertebral bodies;
bone fasteners that secure the said plate to the spine with the diameter of the said bone fasteners being smaller than the diameter of the said bone fastener holes, thereby capable of being placed prior to the said plate so as to provide vertebral body distraction as well as compression with a distraction and compression means;
the said bone fasteners capable of engaging a locking fastener that secures the said bone fastener to the said plate;
the said locking fastener comprising a circumferential superior shoulder that secures the said bone fastener to the said plate in a fixed position

2. The apparatus of claim 1, wherein some of the said bone fastener holes in the said plate are elongated to allow dynamic movement between the said bone fastener and the said plate

3. The apparatus of claim 1, wherein some of the bone fastener holes in the said plate have a socket type configurations that allow bone fasteners with locking fasteners comprising of corresponding ball shaped configurations to be placed at variable angles and also allow dynamic movement between the said bone fastener and the said plate

4. The apparatus of claim 1, wherein said bone fastener has an expandable head incorporated with an expanded head of the said locking fastener

5. The apparatus of claim 1, wherein said bone fastener has an expandable head and tip following engagement by a locking fastener with an expanded head and elongated tip

6. The said locking fastener of claim 4, wherein the shoulders have several ball shaped configurations that settle on corresponding socket type configurations adjacent to the bone fastener holes in the said cervical plate thereby allowing the said bone fastener to be placed at variable angles relative to the said plate as well as allowing the said bone fastener to swivel relative to the said plate

7. The said locking fastener of claim 5, wherein the shoulders have several ball shaped configurations that settle on corresponding socket type configurations adjacent to the bone fastener holes in the said cervical plate thereby allowing the said bone fastener to be placed at variable angles relative to the said plate as well as allowing the said bone fastener to swivel relative to the said plate

8. The apparatus of claim 1, wherein said plate has an elongated bone fastener hole in the middle

9. The apparatus of claim 1, wherein said distraction and compression means are the same, comprising of cannulated tubes with a pin in the center of the said cannulated tube, wherein the said cannulated tube surrounds the bone fastener and the said pin fits into the slot of the said locking fastener in the said bone fastener

10. The said compressor and distractor of claim 9, wherein one or both of the cannulated tube arms have a partial outer tube at the distal ends without a pin, so as to allow tightening of the locking screw with the compressor in place

11. The apparatus of claim 1, wherein said plate has a recess at both longitudinal ends in the middle which allow for placement of distraction screws which can also be used for compression

12. The apparatus of claim 1, wherein said plate also has elongated bone fastener holes at both longitudinal ends in the middle

13. A cervical spine stabilization apparatus comprising:
a fixation plate with a longitudinal axis and two or more bone fastener holes for engaging at least two vertebral bodies;
bone fasteners that secure the said plate to the spine with the diameter of the said bone fasteners being smaller than the diameter of the said bone fastener holes, thereby capable of being placed prior to the said plate so as to provide vertebral body distraction as well as compression with a distraction and compression means;
the said bone fasteners capable of engaging a locking fastener that secures the said bone fastener to the said plate in a fixed position;
the said bone fastener holes of the said plate consisting of a circumferential ridge which secures the said plate to the said bone fastener following placement of the said locking fastener;
the said locking fastener consisting of a circumferential triangular ridge at the head of the said locking fastener that compresses the said circumferential ridge around the said bone fastener hole of the said plate once the said locking fastener is secured to the said bone fastener

14. The apparatus of claim 13, wherein the said locking fastener has an expanded head and the said bone fastener has an expandable head so as to allow expansion of the said bone fastener head following placement of the said locking fastener thereby further securing the said bone fastener to the said plate

15. The apparatus of claim 13, wherein the said locking fastener has an expanded head as well as an elongated tip and the said bone fastener has an expandable head and tip so as to allow expansion of the said bone fastener head and tip following placement of the said locking fastener thereby further securing the said bone fastener to the said plate and the vertebral body

16. The apparatus of claim 13, wherein said distraction and compression means are the same, comprising of cannulated tubes with a pin in the center of the said cannulated tube, wherein the said cannulated tube surrounds the bone fastener and the said pin fits into the slot of the said locking fastener in the said bone fastener

17. The said compressor and distractor of claim 16, wherein one or both of the cannulated tube arms have a partial outer tube at the distal ends without a pin, so as to allow tightening of the locking screw with the compressor in place

18. A method for cervical spine stabilization comprising the steps of:
providing a bone fastener with a head capable of engaging a locking fastener;
securing the said bone fastener into the vertebral bodies;
placing a distractor that engages the bone fasteners and allows distraction of the vertebral bodies for discectomy or corpectomy and arthrodesis;
placing a plate after removal of the distractor with bone fastener holes that allow the said bone fasteners to slide through the said plate bone fastener holes;
placing a compressor that engages the bone fasteners with the plate in place and allows compression of the interbody arthrodesis bone graft;
placement of the bone screw locking fastener after removal of the distractor securing the said plate to the said bone screw fastener
